(19) United States
(12) Patent Application Publication
(10) Pub. No.: US 2022/0415507 A1
(43) Pub. Date: Dec. 29, 2022

(54) METHOD AND SYSTEM FOR TRAINING ARTIFICIAL INTELLIGENCE MODEL FOR ESTIMATION OF GLYCOLYTIC HEMOGLOBIN LEVELS

(71) Applicant: MONORAMA CO., LTD., Seoul (KR)

(72) Inventor: Chang Ho KIM, Seoul (KR)

(21) Appl. No.: 17/780,001

(22) PCT Filed: Aug. 9, 2021
(86) PCT No.: PCT/KR2021/010486

§ 371 Date: May 25, 2022

(30) Foreign Application Priority Data
Apr. 28, 2021 (KR) 10-2021-0055135

(51) Int. Cl. G16H 50/20; G16H 20/10; G16H 50/30

(52) U.S. Cl. CPC G16H 50/20; G16H 20/10; G16H 50/30

(57) ABSTRACT

A method of training an artificial intelligence model for estimating a hemoglobin A1c (HbA1c) level includes collecting patient information including exercise information and bioinformation of a patient, collecting an actual HbA1c level of the patient, converting the collected patient information and actual HbA1c level into a single standardized data structure format, and training an artificial intelligence model using the converted patient information and actual HbA1c level to generate an artificial intelligence model for estimating an HbA1c level. The bioinformation includes at least one of a blood sugar level, a blood pressure, a heart rate, and a menstrual cycle, and the exercise information is generated on the basis of patient life log data acquired by a patient terminal.

### TECHNICAL FIELD

The present invention relates to a diabetic patient management method, and more particularly, to a method and system for training an artificial intelligence model for estimating or predicting a hemoglobin A1c (HbA1c) level and a diabetic patient management method.

### BACKGROUND ART

Blood glucose is combined with a part of hemoglobin in red blood cells that carry oxygen, and a form in which glucose is bound to hemoglobin is called glycated hemoglobin (hemoglobin A1c (HbA1c)).

An HbA1c test is a test for examining how much hemoglobin in red blood cells, which carry oxygen in blood, is glycated and reflects a glucose change for the last two to three months according to the average lifespan of red blood cells. Even people without diabetes necessarily have glucose, and hemoglobin in human bodies is glycated to some degree. Although a normal HbA1c level varies depending on test methods, up to 5.6% is generally considered normal.

In the case of a patient with diabetes, a glucose level in blood is high, and thus glycated hemoglobin, that is, an HbA1c level, is also high. To prevent complications of patients with diabetes, an HbA1c test is necessary, and the Korean Diabetes Association recommends measuring HbA1c every two or three months.

When blood sugar is not controlled, the level of glycated hemoglobin increases. In general, an HbA1c level may reflect a blood glucose concentration of about three months, and thus an HbA1c test is used as a meaningful criterion for evaluating blood sugar control. Accordingly, patients with diabetes visit hospitals every three months to take an HbA1c test, and medical staff decide the treatment direction after seeing this result which reveals the level of blood sugar management during that period of time. When a prescription is given according to a treatment goal and direction set by the hospital, the patient manages diabetes through self-management of medication, diet, exercise, and lifestyle in his or her daily life.

Diabetes is a disease in which medication and daily life management are particularly important, but there is a problem in that the patient manages himself or herself for several months between hospital care, which is not easy, and it is difficult to determine whether the patient manages himself or herself properly.

Self-testing glucometers for blood sugar management are being supplied, but a blood sugar level widely fluctuates due to food consumption and the like. Therefore, it is preferable to manage daily life not on the basis of blood sugar levels but on the basis of an HbA1c level which represents a blood sugar control state of a long period of time (several months). This is the reason that a treatment direction is set on the basis of an HbA1c level in hospitals.

However, HbA1c can be tested only in hospitals, and thus patients vaguely manage themselves without information on their HbA1c levels for several months. Currently, patients with diabetes use a patient notebook or smartphone application to manage their blood sugar. Blood sugar is managed by recording the amount of food, exercise, etc. in the notebook or application.

However, with this method, it is difficult to manage or know an HbA1c level, which is a meaningful criterion for evaluating blood sugar control, and it is difficult for a patient to intermediately check self-management and the like.

### DISCLOSURE

### Technical Problem

The present invention is directed to providing a method and system for training an artificial intelligence model for predicting a hemoglobin A1c (HbA1c) level which is a criterion for managing a patient with diabetes.

The present invention is also directed to providing a system and method for predicting and managing an HbA1c level of a patient.

The present invention is also directed to providing an HbA1c level prediction system and method for establishing a plan for managing a patient with diabetes, providing the corresponding management, and reflecting patient feedback.

### Technical Solution

One aspect of the present invention provides a method of training an artificial intelligence model for estimating a hemoglobin A1c (HbA1c) level.

The method includes collecting patient information including exercise information and bioinformation of a patient, collecting an actual HbA1c level of the patient, converting the collected patient information and actual HbA1c level into a single standardized data structure format, and training an artificial intelligence model using the converted patient information and actual HbA1c level to generate an artificial intelligence model for estimating an HbA1c level.

The bioinformation may include at least one of a blood sugar level, a blood pressure, a heart rate, and a menstrual cycle, and the exercise information may be generated on the basis of patient life log data acquired by a patient terminal.

The patient information may include a degree of compliance with therapeutic intervention, and the therapeutic intervention may include provision of a medication notification message for a prescribed medicine through a user interface of the patient terminal.

The degree of compliance may be calculated on the basis of a response to the medication notification message for the prescribed medication. The response may be input through the user interface of the patient terminal.

Additionally, the response may be generated on the basis of the life log data sensed by the patient terminal.

The patient information may further include at least one of prescription information, physical information, life information, and a degree of compliance with therapeutic intervention, the prescription information may include prescribed medicine information and medication guidance information, and the physical information may include at least one of a height, a weight, and a waist size of the user.

The life information may include at least one of a sleep index and an activity index, and the life information may be generated on the basis of the life log data acquired by the patient terminal possessed by the patient.

The therapeutic intervention may include provision of an exercise recommendation message, and the degree of compliance may be calculated on the basis of a response of the user to the therapeutic intervention.

The patient information may include a degree of compliance with therapeutic intervention, and a chatbot may convert therapeutic intervention information generated on the basis of the patient information and a measured or estimated HbA1c level into a therapeutic intervention message. It is preferable to calculate the degree of compliance on the basis of a time at which a response to the therapeutic intervention message output to the patient terminal is input to the patient terminal. Whether the response is made and the time of the response may be generated on the basis of the life log data sensed by the patient terminal.

Another aspect of the present invention provides a device for training an artificial intelligence model for estimating an HbA1c level, the device including a database unit configured to build a patient database using patient information including exercise information and bioinformation of patients and actual HbA1c levels of the patients and a neural network modeling unit configured to apply the patient information and the actual HbA1c levels included in the patient database to multi-level machine learning and generate an artificial intelligence model for estimating an HbA1c level.

The bioinformation may include at least one of a blood sugar level, a blood pressure, a heart rate, and a menstrual cycle, and the exercise indices may be generated on the basis of patient life log data acquired by patient terminals.

Another aspect of the present invention provides a diabetic patient management method performed by an information processing device (a patient management server), the diabetic patient management method including receiving patient information from a patient device, applying the patient information to an artificial intelligence model, which is trained with the method of claim 1, for estimating an HbA1c level to estimate an HbA1c level, and providing the estimated HbA1c level through a user interface of a patient terminal.

The diabetic patient management method may include establishing a patient management plan on the basis of the collected patient information and a management goal, providing to the patient terminal a therapeutic intervention message for executing the therapeutic intervention according to the established patient management plan, acquiring a response to the therapeutic intervention message, calculating a degree of compliance with the therapeutic intervention on the basis of the response, and the degree of compliance may be determined on the basis of a response time for the therapeutic intervention message output to the user interface of the patient terminal.

A patient management server may provide content including a text message and an image for increasing the degree of compliance with the therapeutic intervention to the patient terminal together with the therapeutic intervention message or after the providing of the therapeutic intervention message.

The patient information may include exercise information and bioinformation, and the bioinformation may include at least one of an actual HbA1c level, a blood sugar level, a blood pressure, a heart rate, and a menstrual cycle.

The exercise information may be an exercise index generated on the basis of patient life log data acquired by the patient terminal, and the exercise index may be a value determined according to an exercise time corresponding to each type of exercise.

The diabetic patient management method may include providing therapeutic intervention on the basis of the patient information, acquiring a response to the therapeutic intervention, calculating a degree of compliance on the basis of the response, and applying the patient information including the degree of compliance to the artificial intelligence model to estimate an HbA1c level.

The response may be automatically generated on the basis of life log data sensed by the patient terminal and transmitted to a server.

### Advantageous Effects

According to exemplary embodiments of the present invention, it is possible to monitor a state of a patient with diabetes in real time and estimate and predict a hemoglobin A1c (HbA1c) level.

According to another aspect of the present invention, it is possible to automate individualized management for patients with diabetes and increase the degree of compliance with the management.

### DESCRIPTION OF DRAWINGS

FIG. 1 is a diagram illustrating a configuration of a diabetic patient management system according to the present invention.

FIG. 2 is a block diagram schematically illustrating a configuration of a diabetic patient management server according to the present invention.

FIG. 3 is a diagram illustrating a data flow between a server (an artificial intelligence training unit) for managing a patient with diabetes, a patient terminal, and a medical staff terminal according to the present invention.

FIG. 4 is a table showing coefficients determined for ranges of blood pressure measurement results.

FIG. 5 shows an example of such a coefficient specification method.

FIG. 6 is a diagram showing a table in which points are given depending on response times to therapeutic intervention to calculate the degree of compliance with the therapeutic intervention and a matrix based on the table.

FIG. 7 illustrates the mathematical relationship between pieces of training data of an artificial intelligence model (cox) for estimating a hemoglobin A1c (HbA1c) level according to the present invention.

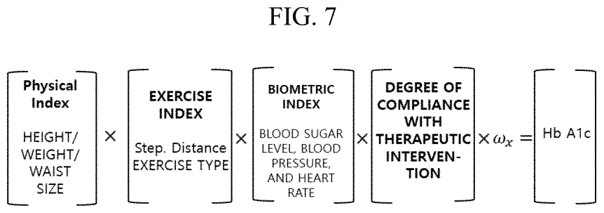

FIG. 8 is a diagram illustrating a data flow between a diabetic patient management server, a patient terminal, and a medical staff terminal according to another embodiment of the present invention.

FIG. 9 is a diagram illustrating a personal disease management (PDM) content provision and response process according to another embodiment of the present invention.

### MODES OF THE INVENTION

Hereinafter, embodiments of the present invention will be described in detail with reference to the accompanying drawings so that those of ordinary skill in the art can readily implement the present invention. However, the present invention can be implemented in various different forms and is not limited to the embodiments described herein. In the drawings, parts unrelated to the description are omitted to clearly describe the present invention. Throughout the specification, like reference numerals refer to like parts.

Throughout the specification, when a part is referred to as “including” a component, unless specifically stated otherwise, this does not mean that other components are excluded and means that other components may be further included.

Hereinafter, embodiments of the present invention will be described in detail with reference to the accompanying drawings. However, the present invention is not limited to or by the embodiments.

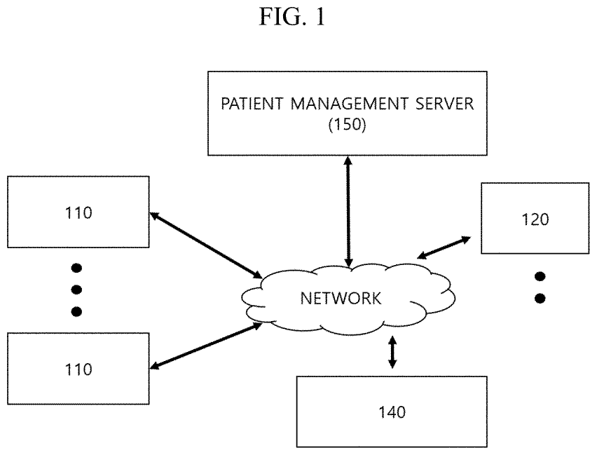

FIG. 1 is a block diagram illustrating a schematic configuration of a diabetic patient management system according to an embodiment of the present invention.

According to FIG. 1, the diabetic patient management system includes a diabetic patient management server 150 and patient terminals 110 possessed by patients with diabetes. The diabetic patient management system may additionally include a medical staff device 120 and/or a third-party device 140. Various terminals and servers of the diabetic patient management system of the present invention can communicate via a communication network in a wired or wireless manner.

The diabetic patient management server 150 may be a single information processing device including a processor, a memory, and a communication module. However, the diabetic patient management server 150 is not limited thereto and may be a system in which a plurality of information processing devices are connected via the communication network.

The patient terminal 110 may be a terminal (e.g., a smartphone, a smart watch, etc.) to which an application for checking health information or a sensor for sensing location information, exercise information, or bioinformation is added. For example, like a smart band, the patient terminal 110 may be worn on a users' body to sense various bioinformation, such as a blood pressure, a heart rate, etc., on the patient, provide patient information to the diabetic patient management server 150, and receive various information from the diabetic patient management server.

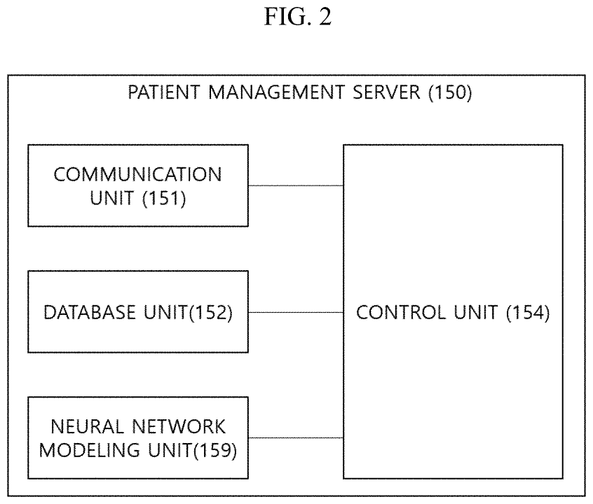

According to FIG. 2, the diabetic patient management server 150 includes an input/output interface (not shown), a communication unit 151, a database unit 152, a neural network modeling unit 159, and a control unit 154. The communication unit 151 may perform wired or wireless communication with the patient terminals 110, the medical staff terminal 120, and/or the third-party device 140 via the communication network.

The database unit 152 includes a patient database in which diabetic patient information is stored. In the neural network modeling unit 159, artificial intelligence is trained using data of the database built by the database unit 152, and an artificial intelligence model which is a neural network model generated as a result is stored. The control unit collects various data from external devices including the patient terminals, controls various data processing in the server, and provides processed data to an external device. Each of the components may be configured in the form of software, hardware, or a combination of software and hardware. Each of the components will be described in detail below.

The diabetic patient management server 150 collects patient data from a patient terminal possessed by a patient with diabetes, provides various information including therapeutic intervention to the patient terminal, collects response or reaction data from the patient terminal, and processes the response or reaction data, thereby helping with self-management of the patient with diabetes. In addition, the diabetic patient management server 150 collects prescription information or various medical information and environmental information from the medical staff device 120 and/or the third-party device 140 and processes the collected information. This will be described below.

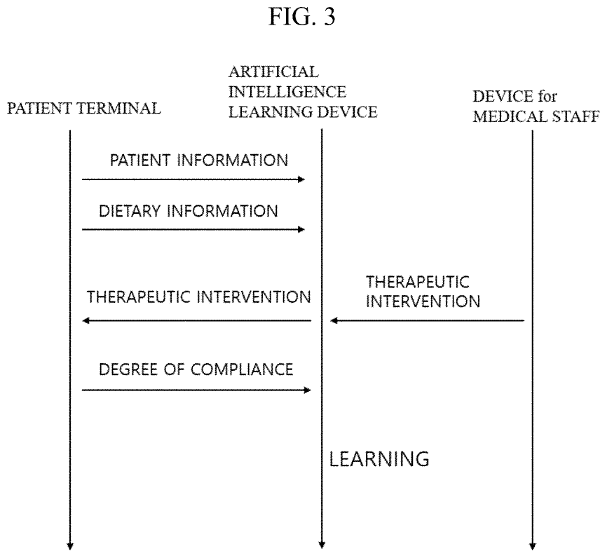

First, a method of training an artificial intelligence model for estimating a hemoglobin A1c (HbA1c) level performed by an artificial intelligence training device will be described in detail with reference to FIG. 3. The training method is implemented by a combination of hardware and a software program of a device for training an artificial intelligence model. An application program stored in a patient terminal interoperates with the software program for data processing.

The artificial intelligence training device is preferably included in the diabetic patient management server 150 but is not limited thereto. The artificial intelligence training device may be implemented as separate hardware and software. A case in which the device for training an artificial intelligence model is included in the diabetic patient management server 150 will be described below.

The device (diabetic patient management server) for training an artificial intelligence model for estimating an HbA1c level according to an embodiment of the present invention includes the input/output interface (not shown), the communication unit 151, the database unit 152 which builds a patient database using patient information and actual HbA1c levels of patients, the neural network modeling unit 159 which generates an artificial intelligence model for estimating an HbA1c level by applying the patient information and the actual HbA1c level data included in the diabetic patient database to multi-level machine learning, and the control unit 154.

The patient information includes patients' exercise information, bioinformation, prescription information, physical information, life information, and degrees of compliance with therapeutic intervention. In addition, the patient information may include the patients' ages, sexes, etc.

The exercise information and life information is generated on the basis of patient life log data acquired by patient terminals. More specifically, a patient terminal may be a smartphone or a smart watch and may collect activity information of a user. The patient terminal may acquire a sleep time, exercise types, type-specific exercise times, calories burned, etc. using life log data, such as the number of steps, a run time, an exercise time, etc., which is sensor data acquired through a gyro sensor, an acceleration sensor, a Global Positioning System (GPS) module, etc. of the smartphone. In some cases, the patient may manually input exercise information or life information, such as one hour of yoga, six hours of meditation, one hour of rest, etc. The patient terminal acquires and transmits such exercise information and life information of the patient to the server 150.

The bioinformation includes at least one of a blood sugar level, a blood pressure, a heart rate, and a menstrual cycle. The blood sugar level may be measured by a separate glucometer, input to the patient terminal by the patient, or measured by an Internet of things (IoT) glucometer having a communication module including a Bluetooth communication module and transmitted to the patient terminal or the server. The bioinformation may further include a blood type, an oxygen saturation, a breathing rate, a breathing capacity, and a body fat measurement result.

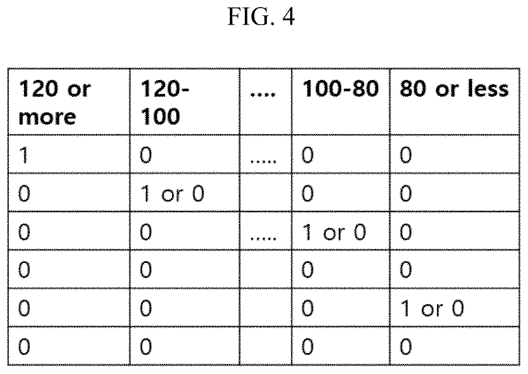

The bioinformation may be measured biological value data, for example, Mar. 1, 2021, a fasting blood sugar level of 100, a normal blood pressure of 100 mmHg to 120 mmHg, and a normal heart rate. Biological value data may be classified into ranges, and coefficients may be separately specified for the ranges. FIG. 4 shows an example in which blood pressure measurement results are classified into ranges and coefficients are specified for the ranges. Different coefficients are specified for the classified ranges depending on patients and dates, and as a result, a biometric index matrix is generated.

Like this, the bioinformation is arranged according to patients (patient identifications (IDs)), dates, and biometric indices and stored in the form of a matrix. Preferably, such data conversion is performed by the database unit 152, and the converted data is structuralized and stored in a patient database.

The prescription information includes the types and amounts of medicines included in a prescription for the patient with diabetes, intake times, and the number of intakes. The prescription information may be input to the patient terminal or the medical staff terminal by the patient or medical staff and transmitted to the server 150. Alternatively, a patient health record (PHR) stored in a hospital device (medical staff device) may be transmitted and provided to the server 150.

The physical information is information related to the patient's body, such as height, weight, waist size, etc. As the physical information, data input to the patient terminal may be received by the server, or, for example, a PHR may be collected by a separate device.

The life information includes a sleep time, a rest time, a travel time, a movement path, etc. The life information may be generated from the life log data acquired by the patient terminal (a wearable device worn by the patient, such as a smart watch or a smartphone). The patient terminal acquires life information, such as the sleep time, the rest time, etc., and the exercise information by processing the life log data including various sensor data of the gyro sensor and the like. The life information may further include smoking and drinking information input to the patient terminal.

The exercise information may be data including the types of exercises and the amount of exercise (time), for example, Mar. 1, 2021, 35 minutes of walking, 30 minutes of running, 0 hours of swimming, and 0 hours of cycling. Coefficients are specified for ranges of exercise-specific exercise times.

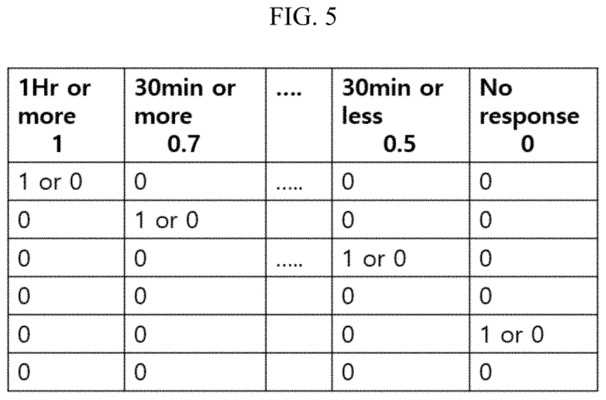

FIG. 5 shows an example of such a coefficient specification method.

Like this, exercise information is arranged according to patients, dates, and exercise-specific exercise times and stored in the form of a matrix. Preferably, such data conversion is performed by the database unit 152, and the converted data is stored in the patient database.

The database unit converts the patient information and actual HbA1c level into a single standardized data structure format, and the neural network modeling unit trains an artificial intelligence model using the converted patient information and actual HbA1c level, thereby generating an artificial intelligence model for estimating an HbA1c level.

A dataset for the training includes an input dataset and an output dataset. The input dataset is the converted patient information, and the output dataset is the actual HbA1c level. However, the actual HbA1c level is measured every two or three months, and thus values reflecting daily changes are input as intermediate HbA1c levels. For example, when a value measured on March 4th is 6.6% and a value measured on June 4th is 6.0%, an actual HbA1c level of April 4th is calculated and input as, for example, 6.0+(31/92)*(6.6−6.0)=6.2%. According to another method, training is performed using an average value in a preprocessing process, the previous value, and the subsequent value to generate artificial intelligence models, and then an artificial intelligence model having a high prediction rate is selected. For example, training is performed using 6.0, 6.3 (an arithmetic average), and 6.6 as HbA1c levels, and then an artificial intelligence model having the highest prediction rate among artificial intelligence models which are generated as a result may be selected.

For the artificial intelligence training, an artificial intelligence algorithm based on, for example, a recurrent neural network (RNN), a convolutional neural network (CNN), a long short-term memory (LSTM), or a deep feedforward network (DFN) may be used.

Meanwhile, for the artificial intelligence training, a response to therapeutic intervention provided through a user interface of the patient terminal may be additionally collected and used.

To this end, the diabetic patient management server 150 may further include a therapeutic intervention unit (not shown), and the therapeutic intervention unit may provide a therapeutic intervention signal based on the patient information and the HbA1c level to the patient terminal. Alternatively, the therapeutic intervention unit may be implemented in the form of an application program stored in the patient terminal. In other words, a therapeutic intervention program generated in an additional server may interoperate with or be included as a part of a diabetic patient management application program.

Therapeutic intervention may include medication guidance, exercise recommendation, a dietary regulation request, a warning, etc. and may be provided through the user interface of the patient terminal. Specifically, therapeutic intervention may be provided through the user interface of the patient terminal in the form of a text message, a social network service (SNS) message, a voice message, a push message, an alarm message of the application program, etc.

The therapeutic intervention unit generates an intervention signal for therapeutic intervention on the basis of the patient information (HbA1c levels, exercise information, bioinformation, prescription information, physical information, life information, dietary information, the degree of compliance with therapeutic intervention, age, and sex).

A therapeutic intervention provision process and a degree-of-compliance calculation process will be described below.

The therapeutic intervention unit generates therapeutic intervention information on the basis of at least one of the patient information and the HbA1c levels, and the control unit controls the communication unit to transmit the therapeutic intervention information to the patient terminal. The degree of compliance with therapeutic intervention is calculated on the basis of a response to the therapeutic intervention information provided to the patient terminal.

The degree of compliance is calculated on the basis of at least one of whether the therapeutic intervention provided through the user interface of the patient terminal is checked, whether a response to the therapeutic intervention is made, and a response time for the therapeutic intervention. The provision of therapeutic intervention and the calculation of the degree of compliance will be described in detail below.

The therapeutic intervention unit generates therapeutic intervention information on the basis of at least one of the converted patient information stored in the database and an estimated HbA1c level estimated by the artificial intelligence model.

Among therapeutic intervention, medication guidance will be described as an example. The prescription information includes types, amounts, intake times, and the number of intakes of medicines for each patient. Therapeutic intervention information is generated according to the prescription information. For example, a request for outputting a medication guidance message or a medication notification message which instructs the patient to take medicine 1 at 8 a.m. may be transmitted to the patient terminal using therapeutic intervention information that medicine 1 is taken after breakfast.

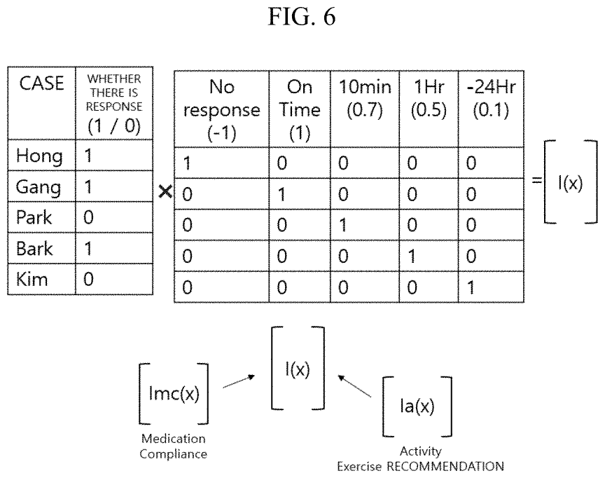

The degree of compliance with the therapeutic intervention is determined on the basis of whether the medication guidance message output through the user interface of the patient terminal is acknowledged and a response time for the medication guidance message. When the message is acknowledged, a score of 1 point is given, and when the message is not acknowledged, a score of 0 points is given. Also, a response to the message (the message includes a response input form or button) may be made. For example, when a response to the message output is immediately made (e.g., within 10 seconds), input after 10 seconds but within 10 minutes, input after 10 minutes but within 1 hour, input after 1 hour but within 24 hours, and input after 24 hours or not input at all, scores of 1, 0.7, 0.5, 0.1, and −1 points may be given, respectively (see FIG. 6). These points may be summed up for each day to calculate a daily degree of compliance or cumulatively summed up to calculate a cumulative degree of compliance. The cumulative degree of compliance is summed from a day that an actual HbA1c level is measured. The higher the daily or cumulative degree of compliance, the higher the degree of compliance.

In addition to medication guidance, therapeutic intervention includes exercise recommendation, dietary regulation, smoking cessation, a controlled drinking request message, etc., and the therapeutic intervention information may be generated on the basis of the estimated HbA1c level and/or the patient information. The generated therapeutic intervention may be converted into a natural language message by a chatbot and provided to the patient terminal.

The degree of compliance with the therapeutic intervention is calculated on the basis of whether the therapeutic intervention message is acknowledged, whether a response is made to the therapeutic intervention, and a response time. Specifically, the degree of compliance with the therapeutic intervention is determined according to whether the therapeutic intervention message output by the patient terminal is acknowledged and a time taken from a time at which the therapeutic intervention message is output until a response is input. The time taken is classified into ranges to which different scores are given, and points are summed up for each day or cumulatively to calculate the degree of compliance.

The calculated degree of compliance is included as a part of the patient information and included in an input dataset for generating an artificial intelligence model.

A method of calculating the degree of compliance includes an operation of providing the therapeutic intervention message to the patient terminal, an operation of receiving an acknowledgement message corresponding to the therapeutic intervention message from the patient terminal, an operation of extracting a response time of the acknowledgement message, an operation of calculating points on the basis of the response time, and an operation of calculating the degree of compliance by summing up the points.

However, the response is not limited to a click or touch on a response button provided through the user interface of the patient terminal. The patient who receives an exercise recommendation message, a walk recommendation message, etc. may exercise without responding to the message through the terminal. In this case, the exercise may be detected by a sensor of the patient terminal possessed by the patient. When the exercise is detected, the patient terminal generates an acknowledge message which includes the exercise and an exercise start time as a response and a response time.

Like this, even without an explicit response to a message of the patient terminal, when a signal corresponding to the therapeutic intervention information is sensed, a response time and a response are generated and stored as an acknowledgement message. In this case, the acknowledgement message may be (exercise, one hour). The degree of compliance calculated according to the above-described method is used as artificial intelligence training data.

A diabetic patient management method according to another embodiment of the present invention will be described.

The diabetic patient management server may perform the diabetic patient management method, and the method may be implemented with a program including a series of instructions.

The diabetic patient management method includes an operation of receiving patient information from a patient device, an operation of estimating an HbA1c level by applying the patient information to an artificial intelligence model for estimating an HbA1c level, and an operation of providing the estimated HbA1c level through a user interface of the patient terminal.

The management method further includes an operation of providing therapeutic intervention on the basis of the patient information, an operation of acquiring a response to the therapeutic intervention, an operation of calculating the degree of compliance on the basis of the response, and an operation of estimating an HbA1c level by applying the patient information including the degree of compliance to the artificial intelligence model.

As described above, the patient terminal 110 may sense various bioinformation, such as a blood sugar value, a blood pressure, a heart rate, a weight, a pulse, an electrocardiogram, etc. When a user consents to a location-based service, the patient terminal 110 may sense the user's movement and generate life information including the user's amount of exercise, sleep time, distance traveled, etc.

The server 150 may receive the patient information from the patient terminal 110 and store the patient information in an internal storage medium, for example, a database, an external storage medium, or a separate cloud. In this case, the server 150 may separately manage a location at which the corresponding user data is stored. The server 150 may store the storage location of the user data in the internal storage medium.

According to the above-described exemplary embodiment of the present invention, it is possible to estimate an HbA1c level of a patient and provide therapeutic intervention through a patient terminal on the basis of the estimated HbA1c level. Since blood is refreshed every three months, an HbA1c test conducted at a hospital is more medically significant than a blood sugar level measured at home every day.

However, an HbA1c level cannot be acquired at home. Accordingly, it is necessary to monitor a patient's life in real time for three months and appropriately estimate an HbA1c level to provide therapeutic intervention through a chatbot. When a reaction (the degree of compliance) to the therapeutic intervention is checked and the degree of compliance is used as partial patient data, that is, training data for an artificial intelligence model for estimating an HbA1c level, it is possible to estimate an HbA1c level with high accuracy. Further, the quality of managing a patient with diabetes can be improved using the HbA1c level as a criterion for self-management.

An artificial intelligence model for estimating an HbA1c level according to an embodiment of the present invention may be installed on a personal computer or various portable terminals, such as a personal digital assistant (PDA), a smartphone, a portable media player (PMP), etc., equipped with a processor (a central processing unit (CPU)) and a memory (a storage) such that an HbA1c level can be estimated directly through the personal terminal without communicating with a server.

Also, a method of estimating an HbA1c level according to the present invention may be implemented in the form of software and configured to operate the above-described personal computer or various portable terminals. The present invention can also be configured in the form of a recording medium on which a program for performing the method of estimating an HbA1c level is recorded.

The present invention is not limited to such a configuration, and any configuration capable of effectively estimating an HbA1c level using the components of the present invention falls within the scope of the present invention.

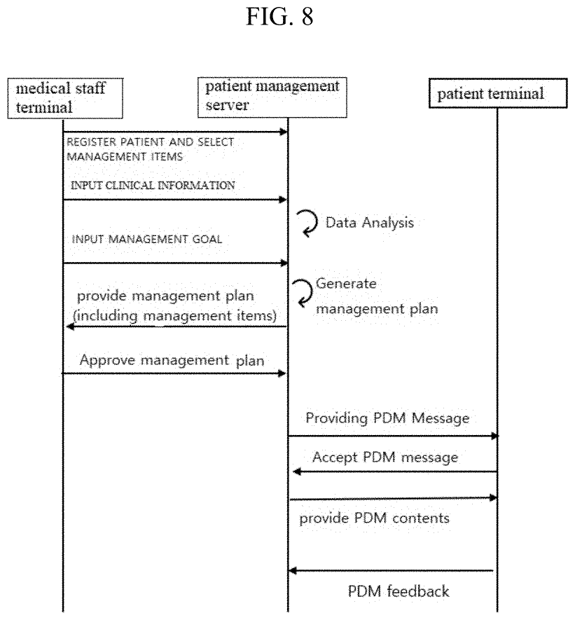

A procedure for establishing a diabetic patient management method according to another embodiment of the present invention will be described below with reference to FIG. 8. Specifically, establishment of a diabetic patient management plan, therapeutic intervention according to the diabetic patient management plan, and feedback on the therapeutic intervention will be described.

In the diabetic patient management method according to the other embodiment of the present invention, a specific patient is registered through a medical staff terminal, and information for selecting diabetes management items is transmitted to a patient management server. Subsequently, when clinical information and a management goal of the patient are transmitted to a patient management server through the medical staff terminal, the patient management server analyzes collected data and generates a patient management plan.

The collected data may be patient information and management items selected by medical staff, and the management items may be, for example, exercise, diet, smoking, and drinking. The management goal input through the medical staff terminal may be, for example, an HbA1c level of 6.5.

The patient management server has the above-described configuration shown in FIG. 2 and further includes a personal disease management (PDM) planning unit. The PDM planning unit generates an individualized management plan for the patient with diabetes on the basis of the patient information, the management items, and the management goal. The management plan may be automatically generated by a prestored program of an algorithm.

The generated management plan is provided to the medical staff terminal. When an approval for the provided management plan is input, automatic management including therapeutic intervention is started by the patient management server. The approval input is made using an approval input method provided to a terminal interface and may be made in various ways such as a button touch, a voice input, etc.

The management plan includes a series of therapeutic intervention information, and provision of a therapeutic intervention message for providing the therapeutic intervention information is basically the same as the above-described process of FIG. 3. Also, artificial intelligence is trained using patient information including the degrees of compliance with therapeutic intervention, and a method of estimating an HbA1c level using the artificial intelligence model formed through the training is the same.

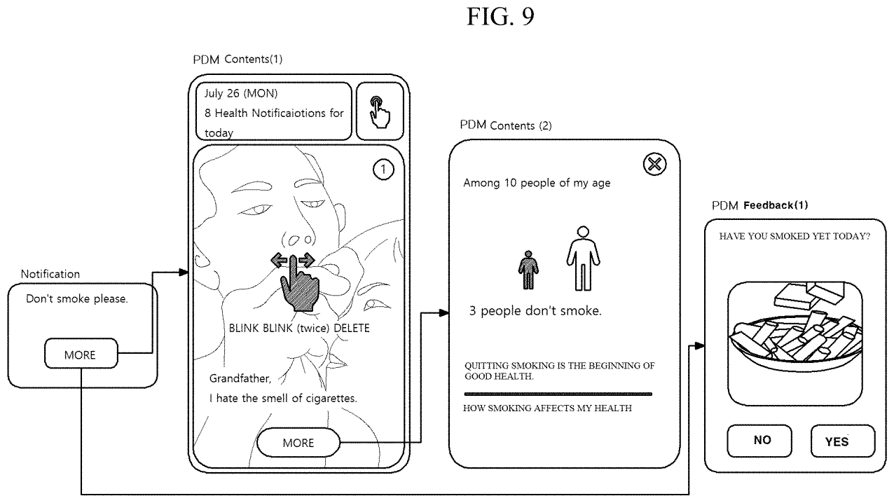

Meanwhile, a therapeutic intervention method according to the other embodiment of the present invention will be described in detail with reference to FIGS. 8 and 9. According to the generated patient management plan, the patient management server provides a PDM message (a therapeutic intervention message) based on the therapeutic intervention information (detailed intervention of a PDM plan) to the patient terminal and outputs the PDM message through an interface of the patient terminal. When the patient accepts the PDM message (touches an Accept button) through the patient terminal, the patient management server provides the PDM content to the patient terminal.

The PDM content includes a text message and an image for increasing the degree of compliance with the therapeutic intervention. Referring to FIG. 9, when a More button is touched after PDM content 1 for preventing smoking is provided to the patient terminal, PDM content 2 may be additionally provided. Such provision of PDM content is optional. Without PDM content, a PDM feedback message for providing an answer to whether the patient has smoked may be directly provided. In this case, the answer, such as yes, no, etc., is the degree of compliance with the therapeutic intervention and may be transmitted to the patient management server according to a response time and response content and used as data for artificial intelligence training.

### INDUSTRIAL APPLICABILITY

The above-described training method provides a system and method for managing a patient with diabetes and can be used in the healthcare industry.

1. A method of training an artificial intelligence model for estimating a hemoglobin A1c (HbA1c) level, the method comprising:
collecting patient information including exercise information and bioinformation of a patient;
collecting an actual HbA1c level of the patient;
converting the collected patient information and actual HbA1c level into a single standardized data structure format; and
training an artificial intelligence model using the converted patient information and actual HbA1c level to generate an artificial intelligence model for estimating an HbA1c level,
wherein the bioinformation includes at least one of a blood sugar level, a blood pressure, a heart rate, and a menstrual cycle, and
the exercise information is generated on the basis of patient life log data acquired by a patient terminal.

2. The method of claim 1, wherein the patient information includes a degree of compliance with therapeutic intervention,
the therapeutic intervention includes provision of a medication notification message for a prescribed medicine through a user interface of the patient terminal, and
the degree of compliance is calculated on the basis of a response to the medication notification message.

3. The method of claim 1, wherein the patient information further includes at least one of prescription information, physical information, life information, and a degree of compliance with therapeutic intervention,
the prescription information includes prescribed medicine information and medication guidance information,
the physical information includes at least one of a height, a weight, and a waist size of the patient,
the life information includes at least one of a sleep index and an activity index,
the life information is generated on the basis of the life log data acquired by the patient terminal possessed by the patient,
the therapeutic intervention includes provision of an exercise recommendation message, and
the degree of compliance is calculated on the basis of a response of the user to the therapeutic intervention.

4. The method of claim 1, wherein the patient information includes a degree of compliance with therapeutic intervention,
a chatbot converts therapeutic intervention information generated on the basis of the patient information and a measured or estimated HbA1c level into a therapeutic intervention message, and
the degree of compliance is calculated on the basis of a time at which a response to the therapeutic intervention message output to the patient terminal is input to the patient terminal.

5. A device for training an artificial intelligence model for estimating a hemoglobin A1c (HbA1c) level, the device comprising:
a database unit configured to build a patient database using patient information including exercise information and bioinformation of patients and actual HbA1c levels of the patients; and
a neural network modeling unit configured to apply the patient information and the actual HbA1c levels included in the patient database to multi-level machine learning and generate an artificial intelligence model for estimating an HbA1c level,
wherein the bioinformation includes at least one of a blood sugar level, a blood pressure, a heart rate, and a menstrual cycle, and
the exercise information is generated on the basis of patient life log data acquired by patient terminals.

6. A diabetic patient management method performed by an information processing device, the diabetic patient management method comprising:
collecting patient information;
applying the patient information to an artificial intelligence model, which is trained with the method of claim 1, for estimating a hemoglobin A1c (HbA1c) level to estimate an HbA1c level; and
providing the estimated HbA1c level through a user interface of a patient terminal.

7. The method of claim 6, wherein the patient information includes exercise information and bioinformation,
the bioinformation includes at least one of an actual HbA1c level, a blood sugar level, a blood pressure, a heart rate, and a menstrual cycle,
the exercise information is an exercise index generated on the basis of patient life log data acquired by the patient terminal, and
the exercise index is a value determined according to an exercise time corresponding to each type of exercise.

8. The method of claim 6, further comprising:
providing therapeutic intervention on the basis of the patient information;
acquiring a response to the therapeutic intervention;
calculating a degree of compliance on the basis of the response; and
applying the patient information including the degree of compliance to the artificial intelligence model to estimate an HbA1c level.

9. The method of claim 6, further comprising:
establishing a patient management plan on the basis of the collected patient information and a management goal;
providing, to the patient terminal, a therapeutic intervention message for executing the therapeutic intervention according to the established patient management plan;
acquiring a response to the therapeutic intervention message; and
calculating the degree of compliance with the therapeutic intervention on the basis of the response,
wherein the degree of compliance is determined on the basis of a response time for the therapeutic intervention message output to the user interface of the patient terminal.

10. The method of claim 9, further comprising providing content including a text message and an image for increasing the degree of compliance with the therapeutic intervention to the patient terminal together with the therapeutic intervention message or after the providing of the therapeutic intervention message.
